(19) United States
(12) Patent Application Publication
(10) Pub. No.: US 2018/0169271 A1
(43) Pub. Date: Jun. 21, 2018

(54) ARMED REPLICATION-COMPETENT ONCOLYTIC ADENOVIRUSES

(71) Applicants: Memgen, LLC, Houston, TX (US); DNAtrix, Houston, TX (US)

(72) Inventors: Mark J. Cantwell, San Diego, CA (US); Winnie M. Chan, La Jolla, CA (US); Brett Ewald, Carlsbad, CA (US); Joan M. Robbins, San Diego, CA (US)

(73) Assignees: Memgen, LLC, Houston, TX (US); DNAtrix, Houston, TX (US)

(21) Appl. No.: 15/847,612

(22) Filed: Dec. 19, 2017

Related U.S. Application Data
(60) Provisional application No. 62/584,008, filed on Nov. 9, 2017.
(60) Provisional application No. 62/437,474, filed on Dec. 21, 2016.

(51) Int. Cl. A61K 48/00; C07K 14/705; C12N 15/86; A61K 38/17; A61K 45/06

(52) U.S. Cl. CPC A61K 48/0058; C07K 14/70575; C12N 15/86; C12N 2830/60; A61K 45/06; C12N 2710/10043; C12N 2710/10033; A61K 38/177

(57) ABSTRACT

Disclosed are replication-competent oncolytic adenoviruses, comprising chimeric human/mouse CD40 ligands. The oncolytic adenoviruses may be replication competent. The chimeric human/mouse CD40 ligand may be MEM40. Also disclosed are methods comprising administering a replication competent oncolytic adenovirus armed with at least one chimeric human/mouse CD40 ligand, for example MEM40, to a patient suffering from a cancer.

This application claims priority from U.S. provisional application 62/437,474, filed on Dec. 21, 2016, and from U.S. provisional application 62/584,008, filed on Nov. 9, 2017, both of which are incorporated herein by reference.

### BACKGROUND OF THE INVENTION

### Field of the Invention

The present invention relates generally to the field of virology, immunology, and medicine. More particularly, it concerns compositions of oncolytic adenovirus vectors for the treatment of cancer.

### Description of Related Art

Oncolytic viruses are a class of cancer therapeutic agents with a dual mechanism of action: 1) tumor cell killing through selective viral replication in tumor cells resulting in direct tumor lysis and 2) induction of systemic anti-tumor immunity by releasing antigens from destroyed tumor cells. Both native and genetically modified viruses are in development. The US FDA approved in 2015 the first oncolytic virus, talimogene laherparepvec (IMLYGIC®, Amgen Inc., Thousand Oaks, Calif.), a genetically modified herpesvirus encoding granulocyte-macrophage colony-stimulating factor (GM-CSF) for the local treatment of melanoma, as described by Kohlhapp et. al. 2016 Clinical Cancer Research. However, melanoma is only one of many types of cancer. Also, GM-CSF is only one of many compounds under investigation for use in cancer therapies. Further, herpesvirus is only one of many viruses under investigation regarding their oncolytic properties.

Accordingly, there remains a need for oncolytic viral vectors for the treatment of cancer.

All of the subject matter discussed in the Background Section is not necessarily prior art and should not be assumed to be prior art merely as a result of its discussion in the Background Section. Along these lines, any recognition of problems in the prior art discussed in the Background Section, or associated with such subject matter, should not be treated as prior art unless expressly stated to be prior art. Instead, the discussion of any subject matter in the Background Section should be treated as part of the inventor's approach to the particular problem, which in and of itself, may also be inventive.

### SUMMARY OF THE INVENTION

The following presents a simplified summary of the disclosure in order to provide a basic understanding of some aspects of the disclosure. This summary is not an exhaustive overview of the disclosure. It is not intended to identify key or critical elements of the disclosure or to delineate the scope of the disclosure. Its sole purpose is to present some concepts in a simplified form as a prelude to the more detailed description that is discussed later.

In some embodiments, the present disclosure relates to oncolytic adenovirus comprising chimeric human/mouse CD40 ligands. The oncolytic adenovirus may be replication competent.

In some embodiments, the present disclosure relates to a method comprising administering to a patient suffering from a tumor a composition comprising an oncolytic adenovirus comprising at least one chimeric human/mouse CD40 ligand.

In either embodiment, the chimeric human/mouse CD40 ligand may be selected from ISF30-ISF41, of which MEM40 (ISF35) is a member.

Transgenes encoding a chimeric human/mouse CD40 ligand selected from ISF30-ISF41, including MEM40 (ISF35) have previously been inserted into a non-oncolytic, non-replicating adenovirus. However, such transgenes have never been engineered into an oncolytic and/or replication-competent adenovirus.

This Summary has been provided to introduce certain concepts in a simplified form that are further described in detail below in the Detailed Description. Except where otherwise expressly stated, this Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

The disclosure may be understood by reference to the following description taken in conjunction with the accompanying drawings, in which:

FIG. 1 schematically represents the construction of Delta-24-RGD-MEM40 (DNX-MEM40) a novel replication competent adenovirus expressing MEM40.

FIG. 2 schematically represents the construction of Delta-24-MEM40, a novel replication competent adenovirus expressing MEM40.

### DESCRIPTION OF ILLUSTRATIVE EMBODIMENTS

Various illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present subject matter will now be described with reference to the attached figures. Various structures are schematically depicted in the drawings for purposes of explanation only and so as to not obscure the present disclosure with details that are well known to those skilled in the art. Nevertheless, the attached drawings are included to describe and explain illustrative examples of the present disclosure. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

While the subject matter disclosed herein is susceptible to various modifications and alternative forms, specific embodiments thereof have been shown by way of example in the drawings and are herein described in detail. It should be understood, however, that the description herein of specific embodiments is not intended to limit the disclosure to the particular forms disclosed, but on the contrary, the intention is to cover all modifications, equivalents, and alternatives falling within the spirit and scope of the disclosure as defined by the appended claims.

### Cancer Overview

“Cancer” refers to a large family of diseases characterized by the uncontrolled growth of cells in a body. Representative forms of cancer include carcinomas, sarcomas, myelomas, leukemias, lymphomas, and mixed types of the above. Further examples include, but are not limited to those discussed in more detail below.

### Adenovirus Overview

“Adenovirus” (Ad) is a large (approximately 36 kb) DNA virus that infects humans, but which also display a broad host range. Physically, adenovirus is an icosahedral virus containing a double-stranded, linear DNA genome. There are approximately 50 serotypes of human adenoviruses, which are divided into six families based on molecular, immunological, and functional criteria. By adulthood, virtually every human has been infected with the more common adenovirus serotypes, the major effect being cold-like symptoms.

Adenoviral infection of host cells results in adenoviral DNA being maintained episomally, which reduces the potential genotoxicity associated with integrating vectors. In addition, adenoviruses are structurally stable, and no genome rearrangement has been detected after extensive amplification. Adenovirus can infect most epithelial cells regardless of their cell cycle stage. So far, adenoviral infection appears to be linked only to mild disease such as acute respiratory disease in humans.

The infectious cycle of the adenovirus takes place in 2 steps: the early phase which precedes initiation of the replication of the adenoviral genome, and which permits production of the regulatory proteins and proteins involved in the replication and transcription of the viral DNA, and the late phase which leads to the synthesis of the structural proteins. The early genes are distributed in 4 regions that are dispersed in the adenoviral genome, designated E1 to E4 (“E” denotes “early”). The early regions comprise at least-six transcription units, each of which possesses its own promoter. The expression of the early genes is itself regulated, some genes being expressed before others. Three regions, E1, E2, and E4 are essential to replication of the virus. Thus, if an adenovirus is defective for one of these functions this protein will have to be supplied in trans, or the virus cannot replicate.

The E1 early region is located at the 5′ end of the adenoviral genome, and contains 2 viral transcription units, E1A and E1B. This region encodes proteins that participate very early in the viral cycle and are essential to the expression of almost all the other genes of the adenovirus. In particular, the E1A transcription unit codes for a protein that transactivates the transcription of the other viral genes, inducing transcription from the promoters of the E1B, E2A, E2B, E3, and E4 regions and the late genes.

The adenovirus enters the permissive host cell via a cell surface receptor, and it is then internalized. The viral DNA associated with certain viral proteins needed for the first steps of the replication cycle enters the nucleus of the infected cells, where transcription is initiated. Replication of the adenoviral DNA takes place in the nucleus of the infected cells and does not require cell replication. New viral particles or virions are assembled after which they are released from the infected cells, and can infect other permissive cells.

The adenovirus is an attractive delivery system. Embodiments of the disclosure can utilize manufacturing process with maximum yields of 1×10⁵ viral particles per cell. The process can be free of or essentially free of protein, serum, and animal derived components making it suitable for a broad range of both prophylactic and therapeutic vaccine products.

If an adenovirus has been mutated so that it is conditionally replicative (replication-competent under certain conditions), a helper cell may be required for viral replication. When required, helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, for example Vero cells or other monkey embryonic mesenchymal or epithelial cells. In certain aspects a helper cell line is 293. Various methods of culturing host and helper cells may be found in the art, for example (Racher, A. J., Fooks, A. R. & Griffiths, J. B. Biotechnol Tech (1995) 9: 169.)

Adenoviruses can be isolated using different methodologies. Most often, after transfection of the Ad genome, adenoviral plaques are isolated from the agarose overlaid cells and the viral particles are expanded for analysis. For detailed protocols the skilled artisan is referred to (Graham, F. L., and Prevec, L. (1991). Manipulation of adenovirus vectors. Methods Mol Biol 7, 109-128.).

Alternative technologies for the generation of adenovirus vectors include utilization of the bacterial artificial chromosome (BAC) system, in vivo bacterial recombination in a recA+ bacterial strain utilizing two plasmids containing complementary adenoviral sequences, and the yeast artificial chromosome (YAC) system (PCT publications 95/27071 and 96/33280, which arc incorporated herein by reference).

Representative examples of adenovirus vectors suitable for use within the present disclosure include those described in US Publication Nos. 2009/0175830, 2014/0377221, 2014/0377294, 2015/0306160, 2016/0289645, and 2016/0143967, and U.S. Pat. Nos. 6,210,946, 6,284,742, 6,312,699, 6,555,368, 6,649,396, 6,815,200, 6,824,771, 6,841,540, 6,955,808, 7,045,348, 7,297,542, 8,168,168 and 9,061,055, all of which are incorporated by reference in their entirety.

### Oncolytic Virus Overview

There are a broad range of oncolytic virus types in development as anti-cancer agents, including adenovirus (see Russell et. al., 2014 Nature Biotechnology and Lawler et. al., 2017 JAMA Oncology).

Multiple biologic properties may be considered in selection or design of a therapeutic oncolytic adenovirus for desired therapeutic activity, including: selective targeting of cancer cells for infection through natural tropism of cell surface proteins or by engineering adenovirus to directly target cancer cells; selective replication in cancer cells; attenuation of viral pathogenesis; enhancing lytic activity; modification of the antiviral immune response that can lead to rapid clearance of adenovirus; and modification of systemic anti-tumor immunity through genetic modification of adenoviruses to incorporate cytokines, immune agonists, or immune checkpoint blockers.

Replication competent oncolytic adenovirus vectors have several properties that make them ideal for therapeutic applications, including infectivity of a broad range of cell and tumor types, infection of non-non-dividing cells, lack of genomic integration, high titers, capacity to carry transgenes, in vitro and in vivo stability, and high levels of expression of transgenes. Adenovirus expression vectors include constructs containing adenovirus sequences sufficient to (a) support packaging of the construct and (b) to ultimately express a recombinant gene construct that has been cloned therein.

Modulation of the biological properties of oncolytic adenoviruses can impact a range of immune interactions that may be beneficial or detrimental in effect on cancer treatment. The interactions depend on the specific tumor, the site and extent of the disease, the immunosuppressive tumor microenvironment, the oncolytic virus platform, the dose, time, and delivery conditions, as well as individual patient responses (see generally Aurelian L. “Oncolytic viruses as immunotherapy: progress and remaining challenges” Onco. Targets Ther. 2016; 9:2627-2637). For example, the presence of adenovirus E3 genes has been reported to increase the oncolytic potency of conditionally replicating adenovirus in vitro and in vivo (see Suzuki K, Alemany R, Yamamoto M, and Curiel DT “The presence of the adenovirus E3 region improves the oncolytic potency of conditionally replicative adenoviruses” Clin. Cancer Res. 2002 November; 8(11):3348-59). In particular, the E3-11.6 kDa Adenovirus Death Protein (ADP) is thought to be required for efficient cell death (see Tollefson A, Ryerse J, and Scaria A, et. al., “The E3-11.6-kDa Adenovirus Death Protein (ADP) is required for efficient cell death: characterization of cells infected with adp mutants,” Virology 1996; 220:152-162). However, for immunotherapeutic approaches to the treatment of cancer, it may be important to balance rapid cell death with sufficient expression of immune modulatory proteins for optimal induction of anti-cancer immune responses. The present disclosure provides such oncolytic adenoviruses.

Members of any of the 57 human adenovirus serotypes (HAdV-1 to 57) may incorporate heterologous nucleic acid encoding an immune cell stimulatory receptor agonist according to the disclosure. Human Ad5 is well characterized genetically and biochemically (GenBank M73260; AC 000008). Thus, in a particular embodiment, the oncolytic adenovirus is a replication competent Ad5 serotype or a hybrid serotype comprising an Ad5 component. The adenovirus may be a wild type strain but may be genetically modified to enhance tumor selectivity, for example by attenuating the ability of the virus to replicate within normal quiescent cells without affecting the ability of the virus to replicate in tumor cells. Non-limiting examples of replication competent oncolytic adenoviruses encompassed by the present disclosure include Delta-24, Delta-24-RGD, ICOVIR-5, ICOVIR-7, ONYX-015, ColoAd1, H101 and AD5/3-D24-GMCSF. Onyx-015 is a hybrid of virus serotype Ad2 and Ad5 with deletions in the E1B-55K and E3B regions to enhance cancer selectivity. H101 is a modified version of Onyx-015. ICOVIR-5 and ICOVIR-7 comprise an Rb-binding site deletion of E1A and a replacement of the E1A promoter by an E2F promoter. ColoAd1 is a chimeric Add11p/Ad3 serotype. AD5/3-D24-GMCSF (CGTG-102) is a serotype 5/3 capsid-modified adenovirus encoding GM-CSF (the Ad5 capsid protein knob is replaced with a knob domain from serotype 3).

Oncolytic adenoviruses injected into a tumor induce cell death and release of new adenovirus progeny that, by infecting the neighbor cells, generates a treatment wave that, if not halted, may lead to the total destruction of the tumor. Significant antitumor effects of Delta-24 have been shown in cell culture systems and in malignant glioma xenograft models. Delta-24-RGD has shown anti-tumor effects in Phase I clinical trials and is currently the subject of additional clinical trials. Although lysis of tumor cells is the main anti-cancer mechanism proposed for Delta-24-RGD oncolytic adenovirus, data from Phase I clinical trials in patients with recurrent glioma and other observations indicate that the direct oncolytic effect may be enhanced by the adenovirus-mediated trigger of anti-tumor immune response.

Within some embodiments of the disclosure, one or more heterologous sequences can be incorporated into a nonessential region of the adenovirus. Within a particular embodiment of the disclosure one or more heterologous sequences can be integrated in place of all or part of the E3 region. Representative examples include cytokines, chemokines and checkpoint inhibitors. Within some embodiments the heterologous sequence code for an OX40 agonist (e.g., OX40L), GITRL anti-PD-1, and/or anti-CTLA-4. Within another embodiment the heterologous nucleic acid sequence encodes an inhibitor of an immune checkpoint protein selected from the group consisting of CTLA4, PD-1, PD-L1, PD-L2, B7-H3, B7-H4, TIM3, GAL9, LAG3, VISTA, KIR, and/or BTLA. Within yet another embodiment the heterologous nucleic acid sequence encodes an agonist of an immune co-stimulatory receptor selected from the group consisting of CD28, OX40 (CD134), glucocorticoid-induced TNF-receptor (GITR), CD137 (4-1BB), herpes virus entry mediator A (HVEM), inducible T-cell costimulator (ICOS or CD278), CD27, CD40, and/or CD226. Representative examples are disclosed in more detail in PCT/US2014/066920 and U.S. Provisional No. 62/342,482, filed May 27, 2016, both of which are incorporated by reference in their entirety.

### DNX-2401

In some embodiments, the replication competent oncolytic adenovirus is Delta-24 or Delta-24-RGD (DNX-2401). Delta-24 is described in U.S. Patent Application Publication Nos. 20030138405, and 20060147420, each of which are incorporated herein by reference. The Delta-24 adenovirus is derived from adenovirus type 5 (Ad-5) and contains a 24-base-pair deletion within the CR2 portion of the E1A gene that encompasses the area responsible for binding Rb protein (nucleotides 923-946) corresponding to amino acids 122 to 129 in the encoded E1A protein (Fueyo J et. al., Oncogene, 19:2-12 (2000)). Delta-24-RGD further comprises an insertion of the RGD-4C sequence (which binds strongly to αvβ3 and αvβ5 integrins) into the HI loop of the fiber knob protein (Pasqualini R. et. al., Nat Biotechnol, 15:542-546 (1997)). The E1A deletion increases the selectivity of the virus for cancer cells; the RGD-4C sequence increases the infectivity of the virus for gliomas and for several other tumors which express low level of adenovirus receptors.

### CD40 Agonist Overview

Though not to be bound by theory, the success of cancer immunotherapy may depend on enhancing tumor-specific CD8+ T cell immunity since CD8+ T cells are strongly associated with direct tumor killing and patient survival. Thus, therapeutic modalities that promote CD8+ T cell responses are a goal in cancer immunotherapy drug development.

The CD40 receptor is a member of tumor necrosis factor receptor family and is expressed by B cells, professional antigen-presenting cells, and non-immune cells and tumors (van Kooten et. al., 2000 Journal of Leukocyte Biology). Activation of tumor-specific T cell responses requires activation of the CD40 receptor on antigen presenting cells. CD4+ T cells enhance CD8+ T cell priming by licensing dendritic cells (DCs) via CD40-CD154 interactions. In addition, CD40-CD154 interactions prevent the CD8+ T cell response from diminishing prematurely. Therefore, CD40 activation through CD154 is a requisite step for inducing effective antigen-specific CD8 T cell immunity against pathogens and tumor (Singh et. al., 2017 Nature Communications). In this regard, CD40-agonist therapies, such as antibodies or cognate CD40 ligand (CD40L) proteins, appear as a promising strategy for cancer immunotherapy.

### ISF35 Overview

ISF35 (which may also be referred to herein as “MEM40”) is a chimeric human/mouse CD40 ligand that has 92% amino acid sequence homology with human CD40L. See, U.S. Pat. No. 7,495,090, herein incorporated by reference). (“CD40 ligand” and “CD40L” may be used interchangeably herein, and may also be referred to as “CD154”). Specifically, domains I, II and III—the regions that contain the intracellular, intra-membrane, and proximal extracellular domains, respectively, of this molecule—have been fully humanized. In domain IV, which contains the CD40 binding portion of the molecule, only those murine domains necessary for optimum CD40 ligand expression in cells are retained. ISF35 (MEM40) is fully humanized at the 3′ end of the molecule where antibody binding neutralizes the activity of the murine CD154 (CD40 ligand) when administered to humans.

Besides MEM40, there is a range of chimeric CD40 ligand constructs (ISF30 through ISF41), MEM40 being a specific chimeric CD40 ligand in the panel with most preclinical and clinical experience.

### Illustrative Embodiments

In some embodiments, the present disclosure relates an oncolytic adenovirus comprising at least one chimeric human/mouse CD40 ligand.

In some embodiments, the present disclosure relates to a replication competent oncolytic adenovirus comprising at least one chimeric human/mouse CD40 ligand. In a particular embodiment, the replication competent oncolytic adenovirus may be genetically modified to incorporate a chimeric human/mouse CD40 ligand transgene.

In some embodiments, the present disclosure relates to a replication competent oncolytic adenovirus comprising a sequence encoding a CD40 agonist operatively linked to a transcriptional control element.

In still other embodiments, the present disclosure relates to replication competent oncolytic adenovirus comprising both a chimeric human/mouse CD40 ligand and one or more additional immunodulatory or therapeutic genes, such as a cytokine (e.g., GM-C SF, TNF□), an interleukin (e.g., IL-2, IL-12), a chemokine (e.g., RANTES), a macrophage inflammatory protein (e.g., MIP-3), a checkpoint inhibitor (e.g., anti-PD-1, anti-CDTA4, and anti-PD-L1), or another immunomodulatory protein (e.g., OX40 ligand).

Genomic regions of the oncolytic adenovirus may be altered for multiple purposes to impart desirable therapeutic properties. Non-limiting examples of therapeutic properties may include enhanced viral replication and spread, enhanced oncolysis, preferential targeting of tumor cells versus normal cells, enhanced immune activation, and protection of adenovirus from the host immune system. Viral regions for the purposes described above may be either eliminated (complete or partial deletions), made non-functional, modified to attenuate function, or substituted by other sequences.

In some embodiments, the present disclosure provides adenovirus with improved capabilities for both immune-mediated and viralytic destruction of tumor cells.

In some embodiments, the present disclosure relates to a method comprising administering to a patient suffering from a tumor a composition comprising a oncolytic adenovirus comprising at least one chimeric human/mouse CD40 ligand.

Any oncolytic adenovirus strain may be used as a starting point for incorporation of the chimeric human/mouse CD40 ligand transgene. In some embodiments, the oncolytic adenovirus is a replication competent human type 5 adenovirus.

Genetic modification of an oncolytic adenovirus to incorporate the chimeric human/mouse CD40 ligand transgene may be performed using techniques known to the person of ordinary skill in the art. The oncolytic adenovirus can contain the chimeric human/mouse CD40 ligand transgene with necessary regulatory elements (cytomegalovirus (CMV) promoter or alternative promoter; polyadenylation domain) to allow for chimeric human/mouse CD40 ligand gene transcription and expression of the chimeric human/mouse CD40 ligand polypeptide in infected cells.

In some embodiments, the chimeric human/mouse CD40 ligand may be selected from the group consisting of ISF30 (SEQ ID NO:1), ISF31 (SEQ ID NO:2), ISF32 (SEQ ID NO:3), ISF33 (SEQ ID NO:4), ISF34 (SEQ ID NO:5), ISF35 (MEM40) (SEQ ID NO:6), ISF36 (SEQ ID NO:7), ISF37 (SEQ ID NO:8), ISF38 (SEQ ID NO:9), ISF39 (SEQ ID NO:10), ISF40 (SEQ ID NO:11), and ISF41 (SEQ ID NO:12).

Polynucleotide sequences encoding the polypeptide sequences of ISF30, ISF32, ISF34, ISF36, ISF38, and ISF40 are disclosed in U.S. Pat. No. 7,928,213, which is hereby incorporated by reference herein. Polynucleotide sequences encoding the polypeptide sequences of ISF31, ISF33, ISF35, ISF37, ISF39, and ISF41 are disclosed in U.S. Pat. No. 7,495,090, which is hereby incorporated by reference herein.

In a particular embodiment, the chimeric human/mouse CD40 ligand incorporated into a replication competent oncolytic adenovirus may be ISF35 (MEM40) (SEQ ID NO:6).

The heterologous chimeric human/mouse CD40 ligand can be inserted at any nonessential location in the oncolytic adenovirus. In some embodiments, the oncolytic adenovirus remains replication competent. In a particular embodiment, the heterologous chimeric human/mouse CD40 ligand nucleic acid is inserted in the E3 region of a replication-competent adenovirus backbone. The E3 region is nonessential for viral replication. The replication-competent adenovirus can comprise a full or partial E3 deletion. In related aspects, the full E3 region is deleted from the replication-competent adenovirus backbone and the chimeric human/mouse CD40 ligand nucleic acid is inserted into a location containing the full E3 deletion.

The oncolytic adenovirus may be genetically modified further to improve one or more properties for use in treatment of cancer, including, selective replication in cancer cells; attenuation of viral pathogenesis; enhancing lytic activity; modification of the antiviral immune response that can lead to rapid clearance of adenovirus; and modification of viral-induced systemic anti-tumor immunity.

In a particular embodiment, the present disclosure provides a Delta-24 or Delta-24-RGD adenovirus comprising a chimeric human/mouse CD40 ligand nucleic acid inserted in place of a partially or completely deleted E3 region, wherein the heterologous nucleic acid comprises a sequence encoding a chimeric human/mouse CD40 ligand transgene.

In some embodiments, the present disclosure relates to a method comprising administering to a patient suffering from a tumor a composition comprising a replication competent oncolytic adenovirus comprising at least one chimeric human/mouse CD40 ligand. In particular embodiments, the oncolytic adenovirus and the at least one chimeric human/mouse CD40 ligand may be as described above.

In some embodiments, the oncolytic adenovirus comprising at least one chimeric human/mouse CD40 ligand may be a Delta-24-MEM40 replication competent adenovirus.

In another embodiment, the oncolytic adenovirus comprising at least one chimeric human/mouse CD40 ligand may be a Delta-24-RGD-MEM40 replication competent adenovirus.

Regardless of replication competent oncolytic adenovirus and chimeric human/mouse CD40 ligand(s) chosen, the replication competent oncolytic adenovirus comprising at least one chimeric human/mouse CD40 ligand may be used for the treatment of cancer, whereby the adenovirus is administered by intratumoral injection. However, other routes of delivery may also be considered, including intravenously, intraperitoneally, intratracheally, intramuscularly, intracranially, endoscopically, intralesionally, percutaneously, subcutaneously, regionally, or by direct injection or perfusion.

Though not to be bound by theory, a replication competent oncolytic adenovirus comprising at least one chimeric human/mouse CD40 ligand, may have dual mechanisms of action: 1) tumor cell killing through selective viral replication of the oncolytic adenovirus in cancer cells and 2) induction of systemic anti-tumor immunity generated by both viral immune induction and CD40 ligand immune activation.

### Regulatory Elements

Expression cassettes included in vectors useful in the present disclosure contain (in a 5′-to-3′ direction) a transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery gathers and integrates the regulatory information conveyed by each element, allowing different genes to evolve distinct, often complex patterns of transcriptional regulation. A promoter used in the context of the present disclosure includes constitutive, inducible, and tissue-specific promoters.

### Promoter/Enhancers

The chimeric human/mouse CD40 ligand nucleic acid expression may be under the control of a promoter functional in mammalian cells, preferably human tumor cells. The chimeric human/mouse CD40 ligand nucleic acid expression may be under the control of a non-adenoviral promoter. In one embodiment, the promoter directing expression of a chimeric human/mouse CD40 ligand is a cytomegalovirus (CMV) promoter.

The expression constructs provided herein comprise a promoter to drive expression of the programming genes. A promoter generally comprises a sequence that functions to position the start site for RNA synthesis. The best example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. These are typically in the region 30 to 110 bp upstream of the start site, although promoters have been shown to contain functional elements downstream of the start site as well. To bring a coding sequence “under the control of” a promoter, one positions the 5-prime end of the transcription initiation site of the transcriptional reading frame “downstream” of (i.e., 3-prime of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be naturally associated with a nucleic acid sequence and obtained by isolating the 5-prime non-coding sequences located upstream of the coding segment and/or exon. Such a promoter can be referred to as “endogenous.” Similarly, an enhancer may be naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other adenovirus, or prokaryotic or eukaryotic cell, and promoters or enhancers not “naturally occurring,” i.e., containing different elements of different transcriptional regulatory regions, and/or mutations that alter expression.

It may be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Non-limiting examples of promoters include early or late viral promoters, such as, SV40 early or late promoters, cytomegalovirus (CMV) immediate early promoters, Rous Sarcoma Virus (RSV) early promoters; and eukaryotic cell promoters.

### Initiation Signals and Linked Expression

A specific initiation signal may also be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would be readily capable of providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements can bypass the ribosome scanning model of 5-prime methylated Cap dependent translation and begin translation at internal sites (Pelletier et. al., 1988 Molecular and Cellular Biology). IRES elements from two members of the picornavirus family (polio and encephalomyocarditis) have been described (Pelletier et. al., 1988 Molecular and Cellular Biology), as well as an IRES from a mammalian message (Macejak et. al., 1991 Nature). IRES elements can be linked to heterologous open reading frames. Multiple open reading frames can be transcribed together, each separated by an IRES, creating polycistronic messages. The IRES element enables each open reading frame to be accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### Methods of Viral Modification

The various genes referred to may be rendered functionally inactive by several techniques well known in the art, including gene deletion(s), substitution(s), or insertion(s). Similarly, the immunomodulatory genes, including MEM40, may be inserted into the viral genome by methods well known to those skilled in the art. These types of modifications in the adenovirus may be made by homologous recombination methods. For example, adenovirus genomic DNA may be transfected together with a plasmid vector comprising mutated sequence flanked by homologous adenovirus sequences, resulting in DNA recombination and substitution of the parent adenovirus genomic DNA region with new mutated sequence region.

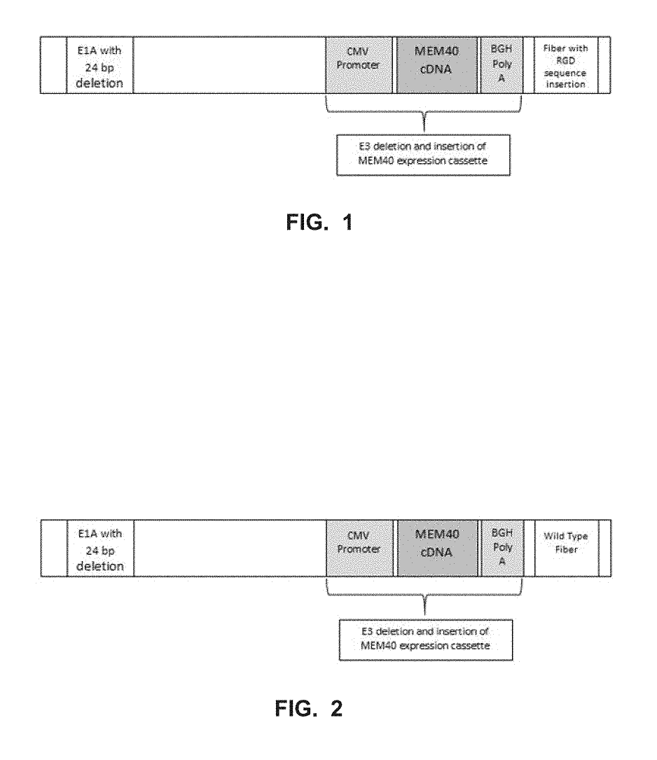

Exemplary constructions of replication competent oncolytic adenoviruses comprising chimeric human/mouse CD40 ligands are shown in FIGS. 1-2.

FIG. 1 schematically represents the construction of Delta-24-RGD-MEM40 (DNX-MEM40), a replication competent adenovirus expressing MEM40. DNX-MEM40 is shown. Briefly, a 24 nucleotide sequence was deleted from the El region. In addition, the E3 region was deleted. A 27 nucleotide sequence coding for an RGD peptide, CDCRGDCFC, was also inserted in the H1 knob domain of the adenovirus fiber sequence. Finally, the MEM40 expression cassette that includes a MEM40 cDNA flanked upstream by a CMV promoter and downstream by a bovine growth hormone polyadenylation signal was inserted into the deleted E3 region.

FIG. 2 schematically represents the construction of Delta-24-MEM40, a replication competent adenovirus expressing MEM40. Delta-24-MEM40 is shown. Construction is similar to DNX-MEM40 (FIG. 1) with the exception that it uses the wild type adenovirus fiber without insertion of the nucleotide sequence coding for an RGD peptide.

### Methods of Screening Adenoviruses for Therapeutic Utility

Oncolytic adenoviruses of the disclosure, or variants or derivatives thereof, can be evaluated for their therapeutic utility by examination of their lytic potential in tumor cells. The tumor cells may include primary tumor cells derived from patient biopsies or surgical resections. Alternatively, the tumor cells may include tumor cell lines. The cytolytic activity of adenoviruses of the disclosure can be determined in tumor cell lines in vitro by infection of cells with serial dilutions of adenovirus and determining the cytolytic potency (e.i. IC50). Particular methods for determining cytolytic activity may include but are not limited MTS, MTT, and ATP colorimetric assays.

The therapeutic index, a comparison of the amount of a therapeutic agent that causes the therapeutic effect to the amount that causes toxicity, of an oncolytic adenovirus of the disclosure may be calculated by comparing the potency of the cytolytic potency of the adenovirus in a tumor cell line with the cytolytic potency in a matched normal cell.

The oncolytic adenoviruses of the disclosure can be further evaluation for therapeutic utility by evaluation of their ability to infect tumor cells and/or normal cells and express a functional chimeric human/mouse CD40 ligand polypeptide encoded by the oncolytic adenovirus. The chimeric human/mouse CD40 ligand expressed on the cell surface of infected cells can be evaluated by flow cytometry using antibodies that specifically recognize human or mouse CD40 antibody binding regions. Chimeric human/mouse CD40 ligand functional activity can also be examined using an in vitro bioassay wherein tumor or normal cells infected with an oncolytic adenovirus encoding a chimeric human/mouse CD40 ligand are mixed with effector cells stably expressing the CD40 receptor fused with a downstream luciferase response element and measuring induced luciferase expression.

The oncolytic adenoviruses of the disclosure can further be evaluated for their ability to target tumor cell growth and the capacity to reduce tumorigenesis or tumor cell burden in mice harboring naturally derived or transplanted tumors in syngeneic or xenogeneic tumor models in mice. Tumor burden as measured by tumor size, immune protection from tumor rechallenge, and animal survival are all possible measures of therapeutic utility and animal tumor models.

### Pharmaceutical Compositions

As noted above, pharmaceutical compositions are provided comprising an adenovirus (as described herein), along with one or more pharmaceutically acceptable diluents, carriers or excipients. Within some embodiments of the disclosure the adenovirus is a replication competent oncolytic adenovirus. Within further embodiments, the adenovirus is Delta-24-MEM40 or Delta-24-RGD-MEM40.

Within certain embodiments of the disclosure, the compositions provided herein can contain pharmaceutically acceptable concentrations of buffering agents, salts, preservatives, as well as other compatible diluents or carriers. The proportion and identity of the pharmaceutically acceptable diluent is selected to be physiologically compatible, and, within particular embodiments, to maintain the viability of the adenovirus. Particular pharmaceutical compositions will be buffered to a suitable pH and be iso-osmotic with physiological fluids.

The pharmaceutical composition provided herein can be prepared by a variety of methods to produce compositions suitable for administration to patients, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

Solutions of the virus may be prepared in a physiologically suitable buffer. Representative buffering agents include, for example, citric acid, sodium citrate, phosphoric acid, potassium phosphate, and various other acids and salts. In some aspects, a mixture of two or more buffering agents is used. The buffering agent or mixtures thereof can be present in an amount of about 0.001% to about 10% by weight of the total composition.

Representative procedures and ingredients for the selection and preparation of suitable formulations are described, for example, in Remington: The Science and Practice of Pharmacy, Lippincott Williams & Wilkins; 21st ed. (May 1, 2005 and in The United States PharmacopE1A: The National Formulary (USP 40-NF 35 and Supplements).

In different embodiments, the composition is administered by injection (subcutaneously, intravenously, intramuscularly, etc.) directly at the disease site, such as a tumor site, or by oral administration, alternatively by transdermal administration.

The forms of the pharmaceutical composition suitable for injectable use include sterile aqueous solutions or dispersions (wherein the term ‘sterile’ does not mean to imply that the adenovirus has been killed, indeed, within particular embodiments as noted above the adenovirus is replication competent. In all cases the form should be sterile and must be fluid to the extent that it is to be deployed through a syringe or catheter.

In addition, the pharmaceutical composition can contain preservatives (to the extent those preservatives do not interfere with the action of the virus. Representative examples of preservatives may include, for example, benzalkonium chloride, methylparaben, propylparaben and sodium benzoate. In further embodiments, a mixture of two or more preservatives is used. The preservative or mixtures thereof can be present in an amount of about 0.0001 to about 2% by weight of the total composition

The adenovirus or pharmaceutical compositions comprising the adenovirus can be packaged as a single vial or package for administration. Kits containing the virus or pharmaceutical composition can also include instructions for preparing and administering the virus or pharmaceutical composition.

### Methods of Treatment and Administration

Within various embodiments of the disclosure, methods are also provided for treating cancer, comprising administering to a subject having cancer adenovirus as described herein. Within further embodiments, the adenovirus is Delta-24-MEM40 or Delta-24-RGD-MEM40. Regardless of the oncolytic adenovirus and chimeric human/mouse CD40 ligand(s) chosen, the oncolytic adenovirus comprising at least one chimeric human/mouse CD40 ligand may be used for the treatment of cancer, whereby the virus is administered by intratumoral injection. However, other routes of delivery may also be considered, including intravenously, intraperitoneally, intratracheally, intramuscularly, intracranially, endoscopically, intralesionally, percutaneously, subcutaneously, regionally, or by direct injection or perfusion.

Within particular embodiments, the cancer is treated utilizing a composition (e.g., a pharmaceutical composition) as described herein. As noted above, the term “cancer” as utilized herein refers to a large family of diseases characterized by the uncontrolled growth of cells in a body. Representative forms of cancer include carcinomas, sarcomas, myelomas, leukemias, lymphomas, and mixed types of the above. Further examples include, but are not limited to bile duct cancer, bladder cancer, brain cancers such as glioblastomas, breast cancer, cervical cancer, CNS tumors (such as a glioblastoma, astrocytoma, medulloblastoma, craniopharyogioma, ependymoma, pinealoma, hemangioblastoma, acoustic neuroma, oligodendroglioma, menangioma, neuroblastoma and retinoblastomas), colorectal cancer, endometrial cancer, hematopoietic cell cancers including leukemias and lymphomas, hepatocellular cancer, kidney cancer, laryngeal cancer, lung cancer, melanoma, oral cancer, ovarian cancer, pancreatic cancer, prostate cancer, squamous cell carcinoma, and thyroid cancer. Cancers may be diffuse (e.g., leukemias), comprise solid tumors (e.g., sarcomas such as fibrosarcoma, myxosarcoma, liposarcoma, chondrosarcoma and osteogenic sarcoma), or some combination of these (e.g., a metastatic cancer having both solid tumors and disseminated or diffuse cancer cells). For example, any cancer patient eligible to receive an autologous or allogeneic stem cell transplant would be considered to be a candidate for this therapy.

In some embodiments, administration can be accomplished by direct administration to a tumor, or to the former site of a tumor (e.g., after surgical resection or an ablation therapy). Administration can be made by direct injection, or by infusion over a selected period of time.

Direct injection into a tumor (intratumoral injection) can be accomplished by a fine catheter or cannula. With certain embodiments, the pharmaceutical compositions provided herein can be delivered by a microelectromechanical (MEMS) system under MR intra-procedural guidance. In particular, intratumoral injection into the brain is accomplished without significant reflux or backflow by using cannula such as Alcyone Lifesciences' Alcyone MEMS Cannula (AMC). Representative examples of devices are described in U.S. Pat. No. 8,992,458 and U.S. Patent Publications 2013/0035660, 2013/0035574 and 2013/0035560, each of which is hereby incorporated by reference in their entirety.

When administered to a subject, an effective amount of a composition as described herein is given in order to treat (e.g., alleviate, improve, mitigate, ameliorate, stabilize, prevent the spread of, slow or delay the progression of or cure) a cancer. For example, it may be an amount sufficient to achieve the effect of reducing the number or destroying cancerous cells or neoplastic cells or by inhibiting the growth and/or proliferation of such cells. In order to be clinically effective, a composition(s) as provided herein could be given once, or, multiple times, depending on the treatment regimen.

A regimen for treatment using the oncolytic adenovirus comprising a gene encoding the at least one chimeric human/mouse CD40 ligand may comprise a single administration or multiple administrations. Multiple administrations may be performed on a recurring schedule and/or in response to one or more indicators of efficacy of one or more prior administrations, or side effects of one or more prior administrations, among others that will be apparent to the person of ordinary skill in the art having the benefit of the present disclosure.

The compositions provided herein can be provided at a variety of concentrations. For example, dosages of adenovirus can be provided which ranges from a dose of greater than about 109 plaque forming units (“pfu”), from between about 102 to above 109 pfu, between about 102 to about 107 pfu, between about 103 to about 106 pfu, or between about 104 to about 105 pfu.

In some embodiments, the oncolytic adenovirus is administered at a dose of 106-1013 plaque forming units (pfu).

The dose of the pharmaceutical composition that is to be used can depend on the particular condition being treated, the severity of the condition, the individual patient parameters including age, physical condition, size and weight, the duration of the treatment, the nature of concurrent therapy (if any), the specific route of administration and other similar factors that are within the knowledge and expertise of the health practitioner. In addition, the dosage may depend on the availability of product.

Though not to be bound by theory, an oncolytic adenovirus comprising at least one chimeric human/mouse CD40 ligand may have dual mechanisms of action: 1) tumor cell killing through selective viral replication of the oncolytic adenovirus in cancer cells and 2) induction of systemic anti-tumor immunity generated by both viral immune induction and CD40 ligand immune activation.

In some embodiments, the composition further comprises a pharmaceutically-acceptable carrier. By “pharmaceutically-acceptable” is meant that the carrier is suitable for use in medicaments intended for administration to a patient. The pharmaceutically-acceptable carrier may vary depending on the route of administration, the storage conditions required for a particular oncolytic adenovirus strain, and other considerations that will be apparent to the person of ordinary skill in the art having the benefit of the present disclosure. In some embodiments, the pharmaceutically-acceptable carrier may be saline.

In some embodiments, the composition may further comprise an adjuvant, such as an enhancer of viral invasion of a tumor cell, an inducer molecule to induce transcription of the gene encoding the at least one chimeric human/mouse CD40 ligand in constructs wherein the gene is under control of a promoter activated by the inducer molecule, or the like.

### Additional Therapies

In some embodiments, the methods may further comprise treatment with a oncolytic adenovirus encoding a chimeric human/mouse CD40 with one or more additional therapies. The additional therapy may be radiation therapy, surgery (e.g., lumpectomy or mastectomy), chemotherapy, gene therapy, DNA therapy, viral therapy, RNA therapy, immunotherapy, biotherapy, bone marrow transplantation, nanotherapy, monoclonal antibody therapy, or a combination of the foregoing. The additional therapy may be in the form of adjuvant or neoadjuvant therapy.

Once the composition is administered to a subject (e.g., a human), the biological activity of the composition can be measured by a variety of methods. Representative parameters which can be assessed include, for example, imaging, and/or by, for example, cytotoxicity assays described in, for example, Kochenderfer et. al., J. Immunotherapy, 32(7): 689-702 (2009), and Herman et. al., J. Immunological Methods, 285(1): 25-40 (2004). In certain embodiments, the biological activity of the compositions provided herein can also be measured by assaying expression and/or secretion of certain cytokines, such as gamma-IFN, IL-2, and TNF. In yet other embodiments of the disclosure the biological activity can be measured by assessing clinical outcome, such as reduction in tumor burden or load.

Any one or all of the one or more additional therapies may be administered before, concurrently with, or after administration of the oncolytic adenovirus.

### Articles of Manufacture of Kits

An article of manufacture or a kit is provided comprising a oncolytic adenovirus encoding a chimeric human/mouse CD40 ligand is also provided herein. The article of manufacture or kit can further comprise a package insert comprising instructions for using the oncolytic adenovirus encoding a chimeric human/mouse CD40 ligand to treat or delay progression of cancer in an individual or to enhance immune function of an individual having cancer. Any oncolytic adenovirus strain encoding a chimeric human/mouse CD40 ligand described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agents include, for example, bottles, vials, bags and syringes.

### EXAMPLES

### Example 1

### Construction and Characterization of pShuttlE3.2-MEM40

DNA of Adenovirus-ISF35 (Lot No. MEM-ADV-FP-009) was isolated using QIAamp DNA Blood Mini Kit (Qiagen) following manufacturer's instructions. A 1.86 kb PCR fragment, containing the cytomegalovirus promoter, MEM40 coding sequence, and bovine growth hormone polyadenylation sequence, was amplified by PCR. The isolated DNA was used as the template and a primer pair containing the BglII and MfeI restriction enzyme sites at the 5′ and 3′ ends, respectively, were used. The resulting PCR product was digested with BglII and MfeI and ligated into pShuttlE3.2 digested with BamHI and EcoRI to insert the MEM40 expression cassette in the place of E3 in the counter-clockwise direction. DH5 cells (Life Technologies) were transformed with the ligation reaction to generate the shuttle vector containing the MEM40 expression cassette (pShuttlE3.2-MEM40). pShuttlE3.2-MEM40 clones were sequenced to confirm the integrity of the inserted MEM40 expression cassette.

### Example 2

### Construction of Delta-24-RGD-MEM40

pShuttlE3.2-MEM40 was digested with NaeI to generate the transfer fragment containing the MEM40 cassette and Kanamycin resistant gene along with the left and right flanking regions for recombination. To construct a recombinant Delta-24-RGD-MEM40, BJ5138 cells (Agilent Technologies) were co-transformed with the transfer fragment and plasmid pVK526, containing the Delta-24-RGD adenoviral backbone, generating pVK526-MEM40/Kan. DH10B cells (Life Technologies) were transformed with the resulting pVK526-MEM40/Kan to isolate more concentrated plasmid DNA. pVK526-MEM40/Kan isolated from DH10B cells were subjected to HindIII restriction enzyme digestion, followed by analysis on 1% agarose gel, to verify the integrity of Delta-24-RGD-MEM40 genome. pVK526-MEM40/Kan was then digested with SwaI to remove the Kanamycin resistant gene and re-circularized by ligation to generate pVK526-MEM40.

To rescue the Delta-24-RGD-MEM40 virus, pVK526-MEM40 was digested with PacI and transfected into A549 cells using Lipofectamine 3000 (Life Technologies) following manufacturer's instructions. Transfected A549 cell monolayers were harvested when cytopathetic effects were observed and cell lysates were used to infect fresh A549 cells to expand the virus. The resulting virus was verified for the presence of the 24 bp deletion in the E1A gene, the RGD insertion in the fiber gene, and the MEM40 expression cassette insertion in the place of the E3 gene.

### Example 3

### Characterization of Delta-24-RGD-MEM40

To characterize Delta-24-RGD-MEM40, A549 cells are infected with Delta-24-RGD-MEM40 for 24 hr and analyzed to confirm cell surface expression of MEM40 by flow cytometry with PE-labelled antibody specific for mouse CD154 (Clone No. MR-1). In addition, the bioactivity of MEM40 is confirmed using the CD40 Bioassay Kit (Promega Corporation).

The particular embodiments disclosed above are illustrative only, as the disclosure may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

### REFERENCES

The following references, to the extent that they provide exemplary procedural or other details supplementary to those set forth herein, are specifically incorporated herein by reference.

Aurelian 2016 Onco. Targets Ther. 9:2627-2637

Fueyo et al., 2000 Oncogene, 19:2-12

Graham et al., 1991 Methods Mol Biol 7:109-128

Herman et al., 2004 J. Immunological Methods, 285(1):25-40

Kochenderfer et al., 2009 J. Immunotherapy 32(7):689-702

Kohlhapp et al. 2016 Clinical Cancer Research 22(5):1048-1054

Lawler et al., 2017 JAMA Oncology 3(6):841-849

Macejak et al., 1991 Nature 353:90-93

Pasqualini et al., 1997 Nat Biotechnol, 15:542-546

PCT Published Patent Application WO 95/27071

PCT Published Patent Application WO 96/33280

Pelletier et al., 1988 Molecular and Cellular Biology 8(3):1103-1112

Racher et al., 1995 Biotechnol Tech 9:169

Remington: The Science and Practice of Pharmacy, Lippincott Williams & Wilkins; 21st ed. (May 1, 2005 and in The United States Pharmacopeia: The National Formulary (USP 40-NF 35 and Supplements).

Remington's Pharmaceutical Sciences 1985 (Remington's Pharmaceutical Sciences, Mack Publishing Company, Easton, Pa., USA)

Roizman 1996 PNAS 93:11307-11312

Russell et al., 2014 Nature Biotechnology 30(7):658-670

Singh et al., 2017 Nature Communications 8(1447):1-10

Suzuki et al., 2002 Clin. Cancer Res. 8(11):3348-59

Tollefson et al., 1996 Virology 220:152-162

U.S. Pat. No. 5,925,565

U.S. Pat. No. 6,210,946

U.S. Pat. No. 6,284,742

U.S. Pat. No. 6,312,699

U.S. Pat. No. 6,555,368

U.S. Pat. No. 6,649,396

U.S. Pat. No. 6,815,200

U.S. Pat. No. 6,824,771

U.S. Pat. No. 6,841,540

U.S. Pat. No. 6,955,808

U.S. Pat. No. 7,045,348

U.S. Pat. No. 7,223,593

U.S. Pat. No. 7,297,542

U.S. Pat. No. 7,495,090

U.S. Pat. No. 7,928,213

U.S. Pat. No. 8,168,168

U.S. Pat. No. 8,992,458

U.S. Pat. No. 9,061,055

U.S. Pat. No. 5,935,819

US Patent Publication No. 2003/0138405

US Patent Publication No. 2006/0147420

US Patent Publication No. 2009/0175830

US Patent Publication No. 2013/0035574

US Patent Publication No. 2013/0035660

US Patent Publication No. 2014/0377221

US Patent Publication No. 2014/0377294

US Patent Publication No. 2015/0306160

US Patent Publication No. 2016/0143967

US Patent Publication No. 2016/0289645

US Patent Publication No. 2013/0035560

van Kooten et. al., 2000 Journal of Leukocyte Biology 67:2-17

1. A composition, comprising;
a replication-competent oncolytic adenovirus containing a heterologous nucleic acid inserted into a nonessential region of the adenovirus genome, said nucleic acid comprising a sequence encoding a CD40 agonist operatively linked to a transcriptional control element.

2. The composition of claim 1, wherein the replication competent oncolytic adenovirus comprises a deletion in part, or all of the E3 gene region.

3. The composition of claim 2, wherein the nucleic acid comprising a sequence encoding a CD40 agonist operatively linked to a transcriptional control element is inserted in the E3 deleted gene region.

4. The composition of claim 3, wherein the nucleic acid comprising a sequence encoding a CD40 agonist operatively linked to a transcriptional control element is inserted in the reverse orientation to the native E3 gene.

5. The composition of claim 1, wherein the CD40 agonist is at least one CD40 ligand (CD40L).

6. The composition of claim 5, wherein the at least one CD40L is a chimeric human/mouse CD40 ligand.

7. The composition of claim 6, wherein the at least one chimeric human/mouse CD40 ligand is selected from the group consisting of ISF30 (SEQ ID NO:1), ISF31 (SEQ ID NO:2), ISF32 (SEQ ID NO:3), ISF33 (SEQ ID NO:4), ISF34 (SEQ ID NO:5), ISF35 (MEM40) (SEQ ID NO:6), ISF36 (SEQ ID NO:7), ISF37 (SEQ ID NO:8), ISF38 (SEQ ID NO:9), ISF39 (SEQ ID NO:10), ISF40 (SEQ ID NO:11), and ISF41 (SEQ ID NO:12).

8. The composition of claim 7, wherein the at least one chimeric human/mouse CD40 ligand is MEM40.

9. The composition of claim 6, wherein the at least one chimeric human/mouse CD40 ligand has at least 90% identity with a ligand selected from the group consisting of ISF30 (SEQ ID NO:1), ISF31 (SEQ ID NO:2), ISF32 (SEQ ID NO:3), ISF33 (SEQ ID NO:4), ISF34 (SEQ ID NO:5), ISF35 (MEM40) (SEQ ID NO:6), ISF36 (SEQ ID NO:7), ISF37 (SEQ ID NO:8), ISF38 (SEQ ID NO:9), ISF39 (SEQ ID NO:10), ISF40 (SEQ ID NO:11), and ISF41 (SEQ ID NO:12).

10. The composition of claim 1, wherein the transcriptional control element operatively linked to the CD40 agonist is a transcriptional promoter.

11. The composition of claim 10, wherein the transcriptional promoter is a cytomegalovirus (CMV) promoter.

12. The composition of claim 1, wherein the replication competent oncolytic adenovirus is a human type 5 adenovirus.

13. The composition of claim 12, wherein the replication competent oncolytic adenovirus is a Delta 24 adenovirus or Delta-24-RGD adenovirus.

14. The composition of claim 1, further comprising a pharmaceutically-acceptable carrier.

15. A method, comprising:
administering to a patient suffering from a cancer a replication competent oncolytic adenovirus containing a heterologous nucleic acid inserted into a nonessential region of the adenovirus genome, said nucleic acid comprising a sequence encoding a CD40 agonist operatively linked to a transcriptional control element.

16. The method of claim 15, wherein the replication competent oncolytic adenovirus comprises a deletion in part, or all of the E3 gene region.

17. The method of claim 16, wherein the nucleic acid comprising a sequence encoding a CD40 agonist operatively linked to a transcriptional control element is inserted in the E3 deleted gene region.

18. The method of claim 17, wherein the nucleic acid comprising a sequence encoding a CD40 agonist operatively linked to a transcriptional control element is inserted in the reverse orientation to the native E3 gene.

19. The method of claim 15, wherein the replication competent oncolytic adenovirus is a Delta 24 adenovirus or Delta-24-RGD adenovirus.

20. The method of claim 15, wherein the CD40 agonist is at least one CD40 ligand (CD40L).

21. The method of claim 20, wherein the at least one CD40L is a chimeric human/mouse CD40 ligand.

22. The method of claim 21, wherein the at least one chimeric human/mouse CD40 ligand is selected from the group consisting of ISF30 (SEQ ID NO:1), ISF31 (SEQ ID NO:2), ISF32 (SEQ ID NO:3), ISF33 (SEQ ID NO:4), ISF34 (SEQ ID NO:5), ISF35 (MEM40) (SEQ ID NO:6), ISF36 (SEQ ID NO:7), ISF37 (SEQ ID NO:8), ISF38 (SEQ ID NO:9), ISF39 (SEQ ID NO:10), ISF40 (SEQ ID NO:11), and ISF41 (SEQ ID NO:12).

23. The method of claim 22, wherein the at least one chimeric human/mouse CD40 ligand is MEM40.

24. The method of claim 21, wherein the at least one chimeric human/mouse CD40 ligand has at least 90% identity with a ligand selected from the group consisting of ISF30 (SEQ ID NO:1), ISF31 (SEQ ID NO:2), ISF32 (SEQ ID NO:3), ISF33 (SEQ ID NO:4), ISF34 (SEQ ID NO:5), ISF35 (MEM40) (SEQ ID NO:6), ISF36 (SEQ ID NO:7), ISF37 (SEQ ID NO:8), ISF38 (SEQ ID NO:9), ISF39 (SEQ ID NO:10), ISF40 (SEQ ID NO:11), and ISF41 (SEQ ID NO:12).

25. The method of claim 15, wherein the transcriptional control element operatively linked to the CD40 agonist is a transcriptional promoter.

26. The method of claim 25, wherein the transcriptional promoter is a cytomegalovirus (CMV) promoter.

27. The method of claim 15, wherein the oncolytic adenovirus is administered in a composition comprising a pharmaceutically-acceptable carrier.

28. The method of claim 15, wherein the patient has a cancer selected from primary or metastatic cancer.

29. The method of claim 15, wherein the patient has brain cancer or bladder cancer.

30. The method of claim 15, wherein the oncolytic adenovirus is administered intratumorally, intravenously, intraperitoneally, intratracheally, intramuscularly, intracranially, endoscopically, intralesionally, percutaneously, subcutaneously, regionally, or by direct injection or perfusion.

31. The method of claim 15, wherein the oncolytic adenovirus is administered once or multiple times.

32. The method of claim 31, wherein the replication competent oncolytic adenovirus is administered at a dose of 10⁶-10¹³ plaque forming units (pfu).

33. The method of claim 15, further comprising:
administering at least one additional therapeutic agent.

34. The method of claim 33, wherein the at least one additional therapeutic agent is chemotherapy, immunotherapy, surgery, radiotherapy, viral therapy, or biotherapy.

35. The method of claim 34, wherein the at least one additional therapeutic agent is administered to the patient before administration of the replication competent oncolytic adenovirus.

36. The method of claim 34, wherein the at least one additional therapeutic agent is administered to the patient at the same time as administration of the replication competent oncolytic adenovirus.

37. The method of claim 34, wherein the at least one additional therapeutic agent is administered to the patient after administration of the replication competent oncolytic adenovirus.

38. The method of claim 15, wherein the patient is a human.


SEQUENCE LISTING (12 sequences)

SEQ ID NO: 1 - DNA, 783 bp, Artificial Sequence
  chimeric human/mouse CD40 ligand
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 2 - DNA, 759 bp, Artificial Sequence
  chimeric human/mouse CD40 ligand
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 3 - DNA, 783 bp, Artificial Sequence
  chimeric human/mouse CD40 ligand
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 4 - DNA, 759 bp, Artificial Sequence
  chimeric human/mouse CD40 ligand
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctgttcacttgggcggagtgtttgaattacaaccaggtgcttcggtgtttgtcaatgtgactgatccaagccaagtgagccatggcactggcttcacgtcctttggcttactcaaactctga

SEQ ID NO: 5 - DNA, 783 bp, Artificial Sequence
  chimeric human/mouse CD40 ligand
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 6 - DNA, 759 bp, Artificial Sequence
  chimeric human/mouse CD40 ligand
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 7 - DNA, 783 bp, Artificial Sequence
  chimeric human/mouse CD40 ligand
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcgtgttttgtcaatgtgactgatccaagccaagtgagccatggcactggcttcacgtcctttggcttactcaaactctga

SEQ ID NO: 8 - DNA, 759 bp, Artificial Sequence
  chimeric human/mouse CD40 ligand
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggaggcttcgagtcaagccccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 9 - DNA, 783 bp, Artificial Sequence
  chimeric human/mouse CD40 ligand
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 10 - DNA, 759 bp, Artificial Sequence
  chimeric human/mouse CD40 ligand
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 11 - DNA, 783 bp, Artificial Sequence
  chimeric human/mouse CD40 ligand
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcggtgtttgtcaatgtgactgatccaagccaagtgagccatggcactggcttcacgtcctttggcttactcaaactctga

SEQ ID NO: 12 - DNA, 759 bp, Artificial Sequence
  chimeric human/mouse CD40 ligand
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagatgaggatcctcaaattgcagcacacgttgtaagcgaagccaacagtaatgcagcatccgttctacagtgggccaagaaaggatattataccatgaaaagcaacttggtaaccctggaaaatgggaaacagctgacggttaaaagacaaggactctattatatctatgctcaagtcaccttctgctctaatcgggagccttcgagtcaacgcccattcatcgtcggcctctggctgaagcccagcagtggatctgagagaatcttactcaaggcggcaaatacccacagttccgccaagccttgcgggcagcagtctattcacttgggcggagtgtttgaattacaaccaggtgcttcggtgtttgtcaatgtgactgatccaagccaagtgagccatggcactggcttcacgtcctttggcttactcaaactctga